(19) United States
(12) Patent Application Publication
(10) Pub. No.: US 2025/0256003 A1
(43) Pub. Date: Aug. 14, 2025

(54) METHODS AND SYSTEMS FOR SCENT PRESENTATION

(71) Applicant: Sciance Lab 3 Inc., Dana Point, CA (US)

(72) Inventor: Young A. Tang, Downey, CA (US)

(21) Appl. No.: 19/053,062

(22) Filed: Feb. 13, 2025

Related U.S. Application Data
(60) Provisional application No. 63/553,000, filed on Feb. 13, 2024.

(51) Int. Cl. A61L 9/12; A61L 9/03; F24F 8/50

(52) U.S. Cl. CPC A61L 9/125; A61L 9/035; A61L 2209/11; A61L 2209/132; A61L 2209/135; F24F 8/50

(57) ABSTRACT

A scent presentation device, the device comprising: a plurality of scent reservoirs configured to contain a liquid; at least one liquid dispersion mechanism configured to disperse liquid into an environment; and a control module configured to cause the at least one liquid dispersion mechanism to disperse liquid from a first scent reservoir of the plurality of scent reservoirs for a first duration, and to disperse liquid from a second scent reservoir of the plurality of scent reservoirs for a second duration.

This application claims the benefit of U.S. Provisional Application No. 63/553,000, filed Feb. 13, 2024, which is incorporated herein by reference in its entirety.

### BACKGROUND

### Field of the Invention

The technology described herein relates to scent dispersion.

### Background Art

Environmental enrichment has been shown to have a positive impact on cognitive function such as in ameliorating cognitive decline in various animal models. Enriching an environment can be accomplished in a variety of ways including, but not limited to, the introduction of various objects, sounds, smells, colors, animals, etc. Thousands of research papers reaching this conclusion have been published and the cognitive benefits have been shown to reduce or overcome animal models of human neurological disorders such as Alzheimer's disease, memory loss, vascular dementia, neuronal death in aging, traumatic brain injury, head injury, Parkinson's disease, seizures, stroke, multiple sclerosis, anxiety, autism, ADHD, Huntington's disease, Down Syndrome, stress, depression, cerebral palsy, chemo-brain, schizophrenia, prenatal alcohol syndrome, lead exposure, addiction and cancer, to name a few. Noticeable behavioral changes have been observed in children with autism after as little as six months of environmental enrichment exercises.

Scent is one medium by which a person's environment may be enriched. A scent may be presented through aerosolization, diffusion, dispersion, and/or nebulization of a medium such as a scent-bearing essential oil or oil mixture, for example.

Aerosolization involves converting substances into aerosols, which are fine particles or droplets capable of being suspended in air. This process can include diffusion, dispersion, and nebulization.

Diffusion is the process by which aerosol particles move from areas of higher concentration to areas of lower concentration, tending towards a uniform distribution within the aerosol. This principle leads to aerosolized particles being evenly dispersed in their environment.

Dispersion refers to the manner in which aerosol particles are spread through a medium, influenced by factors such as air currents and the physical characteristics of the particles, including size and density.

Nebulization involves converting liquid substances into a fine mist. This method is particularly relevant in medical applications, where it facilitates the direct delivery of medication to a patient's respiratory system. Nebulization enables precise targeting to the lungs, improving the efficacy of treatments.

### BRIEF DESCRIPTION OF THE DRAWINGS/FIGURES

FIG. 1 illustrates an ultrasonic diffuser according to an embodiment.

FIG. 2 illustrates a nebulizing diffuser according to an embodiment.

FIG. 3 illustrates an evaporative diffuser according to an embodiment.

FIG. 4 illustrates a heat diffuser according to an embodiment.

FIG. 5 illustrates an ultrasonic nebulizer according to an embodiment.

FIG. 6 illustrates a mesh nebulizer according to an embodiment.

FIG. 7 illustrates a jet nebulizer according to an embodiment.

FIG. 8 illustrates a system for scent dispersion, according to an embodiment.

FIG. 9 illustrates a system for scent dispersion, according to an alternative embodiment.

Further embodiments, features, and advantages of the present invention, as well as the operation of the various embodiments of the present invention, are described below with reference to the accompanying drawings.

### DETAILED DESCRIPTION

A preferred embodiment of the present invention is now described with reference to the figures. While specific configurations and arrangements are discussed, it should be understood that this is done for illustrative purposes only. A person skilled in the relevant art will recognize that other configurations and arrangements can be used without departing from the spirit and scope of the invention. It will be apparent to a person skilled in the relevant art that this invention can also be employed in a variety of other systems and applications.

Of the various stimulants used in environmental enrichments, it has been found that cognition is strongly associated with olfaction. Olfaction is the only sense that has a large, direct pathway to the cognitive and emotional areas of the brain. Loss of olfaction precedes the memory loss from aging and from all forms of dementia. Olfactory loss triggers a massive loss of neurons in the brain. After the age of 50, it has been found that olfactory ability accurately predicts all-cause mortality within the next five years.

Biologically, olfactory stimulation activates the entorhinal cortex. The entorhinal cortex diminishes with age and other factors such as Alzheimer's and other forms of dementia. As the entorhinal cortex diminishes, it releases the dentate gyrus and CA3 (hippocampal subfields) from inhibition, thereby interfering with memory. Thus, a restoration of olfaction should increase neurogenesis and/or neural complexity in the entorhinal cortex, thereby normalizing dentate/CA3 activity and restoring memory.

The systems and apparatuses described herein may be used for multiple purposes, including the therapeutic purposes noted above. The scent presentation device/system (or dispersion device/system) described herein can use various types of mechanisms such as a diffusion mechanism, dispersion mechanism, nebulization mechanism, evaporative mechanism, or a combination thereof, for example and without limitation. Hereinafter, the term “dispersion mechanism” can include one or more of the above mechanisms.

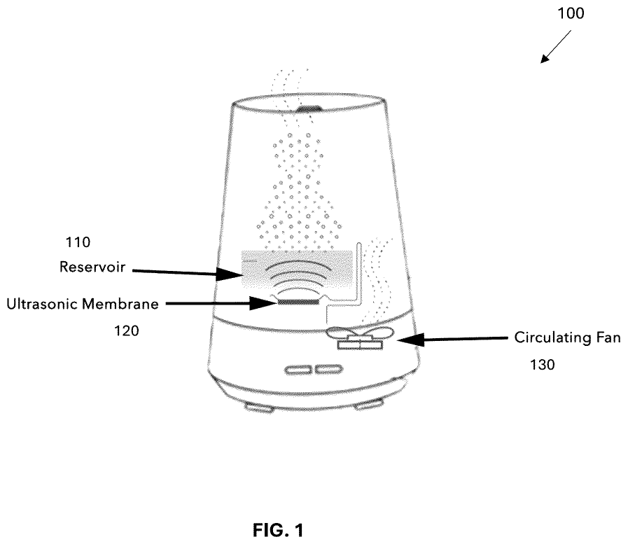

A scent presentation device/system can use an ultrasonic diffuser as a dispersion mechanism, employing ultrasonic vibrations to create a fine mist from essential oil(s) or a mixture of water and essential oil(s). The mist may then be released into the air, adding humidity and dispersing the scent. An embodiment of an ultrasonic diffuser is shown in FIG. 1. A scent reservoir 110 may contain a scent bearing liquid, such as a mixture of water and essential oils. An ultrasonic membrane 120 is made to vibrate to create acoustic waves in the liquid, creating a mist that is propelled out of the device by a forced air mechanism such as a fan 130.

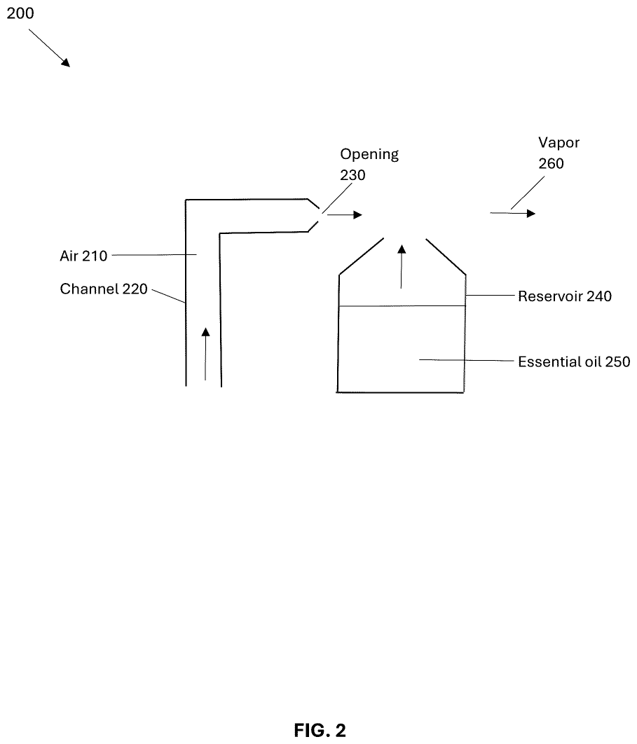

The scent presentation device/system can alternatively use a nebulizing diffuser as a dispersion mechanism, which works by using pressurized air to atomize essential oil(s), either pure or in a mixture, turning them into a fine mist without the use of heat or water. An embodiment is shown in FIG. 2. Here air 210 may be propelled through a channel 220 and an opening 230. A reservoir 240 may hold a scent-bearing liquid such as an essential oil or mixture thereof (250). The propelled air results in lower air pressure at the opening of reservoir 240, drawing evaporated oil 250 from the reservoir 240 in the form of vapor 260, which is then dispersed.

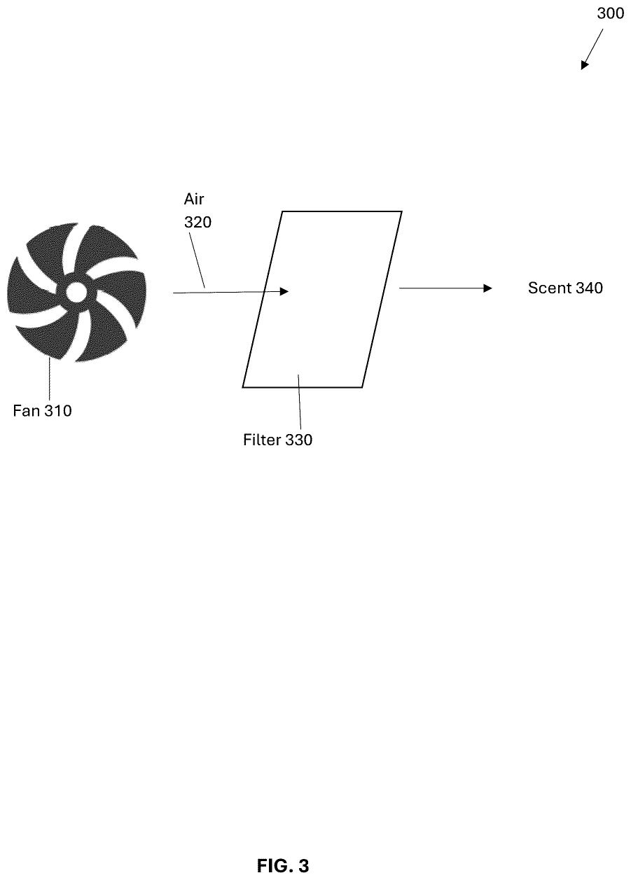

The scent presentation device/system can alternatively use an evaporative diffuser as a dispersion mechanism, as illustrated in FIG. 3 according to an embodiment. This embodiment may use a fan 310 to move air over a pad or through a filter 330 where the pad or filter holds one or more essential oils or a mixture thereof for example. This may evaporate the essential oils from the pad or filter. Alternatively the air stream may evaporate oil droplets exposed to the airstream (with no carrier medium). In any case, as the oil evaporates, the fan 310 may blow the scent into the air. Evaporative diffusion may be suitable for personal use or in volumetrically smaller settings, for example.

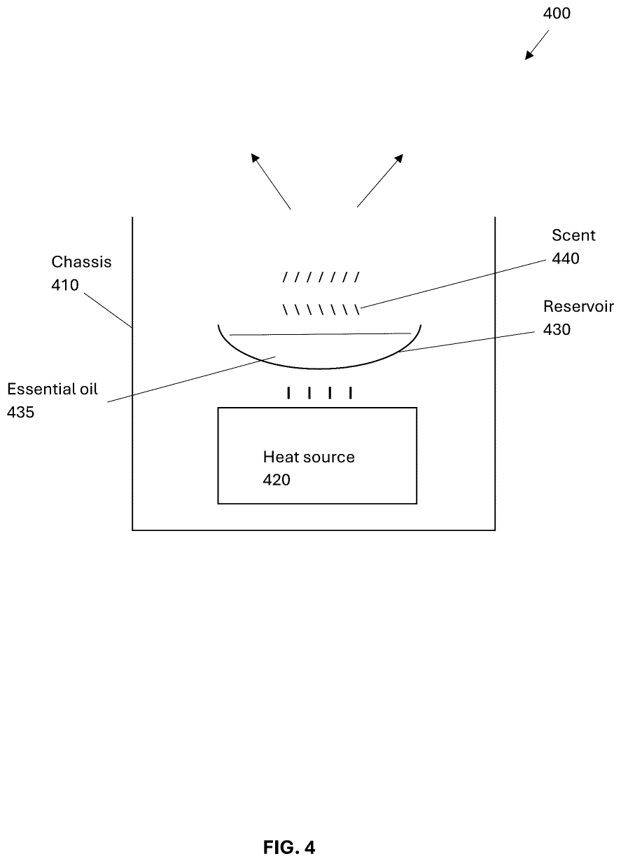

The scent presentation device/system can use a heat diffuser as the dispersion mechanism, to heat essential oil(s), causing them to evaporate and disperse their scent into the air as shown in the embodiment of FIG. 4. A heat source 420 is shown in a chassis 410, and heats a reservoir 430 containing a scent bearing liquid such as an essential oil or mixture thereof (435). The resulting vapor with scent 440 is thereby released.

The scent presentation device/system can use various types of nebulizers as dispersion mechanisms.

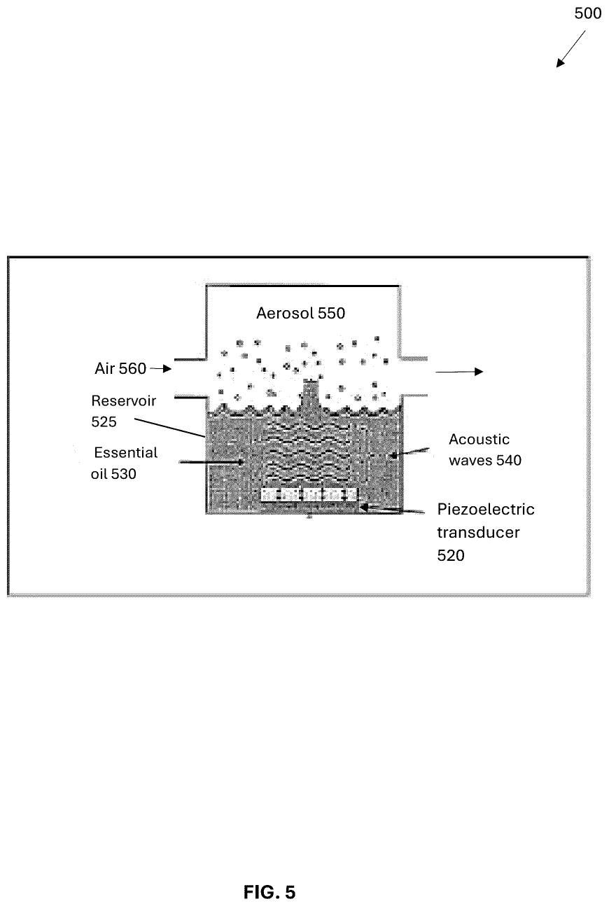

The scent presentation device/system can use an ultrasonic nebulizer as a dispersion mechanism as shown in FIG. 5 according to an embodiment. Here, a piezoelectric transducer 520 may create high-frequency ultrasonic vibrations in an essential oil or a mixture thereof (530) in a reservoir 525. Resulting acoustic waves 540 generate a mist or aerosol droplets 550 from the liquid 530. The aerosol 550 may then be pushed out of the mechanism by an air current 560. The efficiency of ultrasonic nebulizers in creating a fine mist rapidly is notable, although the thermal effect of the process on medication stability warrants consideration for thermolabile substances.

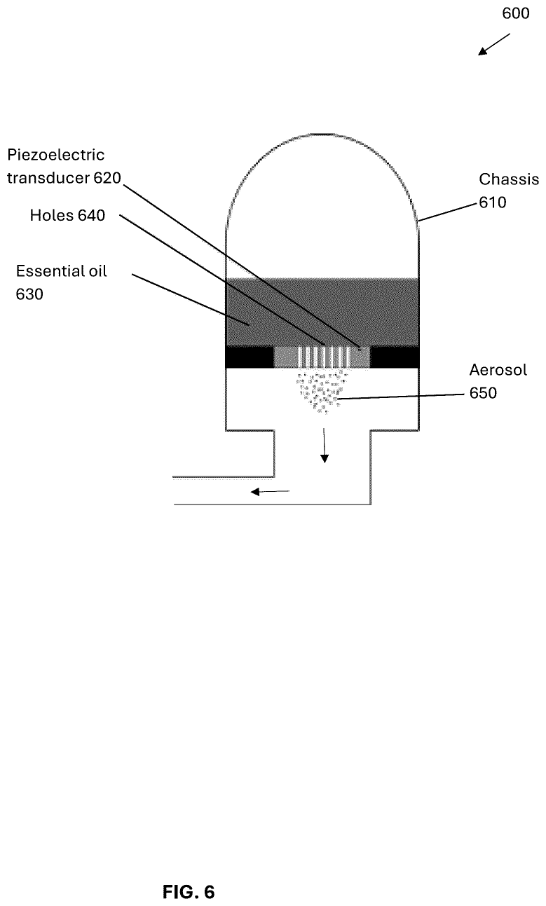

The scent presentation device/system can use a mesh nebulizer as a dispersion mechanism. This embodiment, shown in FIG. 6, uses a mesh or membrane with precisely sized openings (holes 640), through which the liquid 630 (e.g., essential oil(s) or water-oil mixture) is forced under pressure, creating a fine mist or aerosol. This category of systems can be subdivided into active and passive systems. Active mesh nebulizers employ a vibrating element (e.g., driven by a piezoelectric transducer 620) to propel the liquid through the mesh, whereas passive systems rely on negative pressure to draw the liquid across. In either case, an aerosol 650 results. The precision engineering of the mesh ensures consistent droplet size, making these devices highly efficient for delivery with minimal residue.

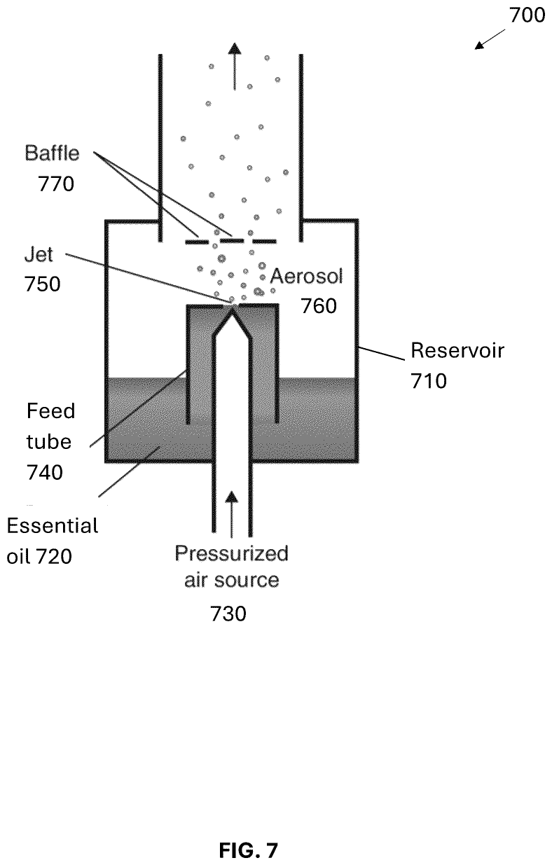

The scent presentation device/system can alternatively use a jet nebulizer as the dispersion mechanism as shown in FIG. 7. This operates on the principle of pressurized air flowing through a narrow orifice, converting a liquid into aerosol droplets. This process may involve a Venturi system, where the high velocity of air creates a negative pressure that draws the liquid from the reservoir, atomizing it into a breathable mist. In the figure, a reservoir 710 holds an essential oil or oil/water mixture 720. A pressurized air source 730 pushes air into the device through a feed tube 740 to produce a jet 750. This creates a low pressure area drawing the oil up and creating an aerosol 760. The aerosol may pass through a baffle 770 before being released.

A scent presentation device/system can be incorporated into an HVAC system. The dispersion system can be integrated with a building's heating, ventilation, and air conditioning (HVAC) system to disperse scents throughout large areas or entire buildings efficiently. The dispersion system can use cold-air diffusion technology to atomize essential oils into a fine, dry mist that is then distributed evenly through the HVAC ducts. This method is ideal for large spaces such as whole-house, offices, and hotels. Similar to HVAC scenting systems but on a smaller scale, cold-air diffusers also use cold-air diffusion technology to atomize essential oils without heat, preserving their therapeutic properties. These standalone units are suitable for medium to large spaces that require consistent scent coverage without connection to the HVAC system.

In an embodiment, a scent presentation system may be integrated into an HVAC system and controlled by smart home technology. This would allow scent presentation to occur according to a schedule that is defined by a user and programmed into the smart home system. Further, a scent dispersion system may be configured to present a scent, pause presentation, then present a different scent (discussed below). In this case, a fan in the HVAC system may be activated during the interval in which presentation is paused, thereby clearing the first scent before the second scent is presented.

It should be noted that the scent presentation system can use any type of aerosolization and evaporation technologies described herein.

### Pyramid Model

The variety of scents may be modeled as a layered pyramid. The pyramid model may be divided into three main categories or layers of notes, each representing a specific phase of a fragrance's life. These layers are:

Top Notes (Head Notes): These are the initial scents that are perceived immediately upon application of a perfume, for example. Top notes are usually light, fresh, and volatile, evaporating quickly. They give the first impression of the fragrance but last for a short duration, typically ranging from a few minutes to an hour. Common top notes include citrus (like lemon and bergamot), light fruits (such as berries and grapes), and herbs (like lavender and rosemary).

Middle Notes (Heart Notes): After the top notes dissipate, the middle notes become noticeable. They are considered the heart of the fragrance, representing the main elements of the scent composition. Middle notes are more mellow and rounded than top notes and are perceived during the majority of the fragrance's wear on the skin. They typically last longer than top notes but are less volatile, usually lasting several hours. Common middle notes include floral (like rose and jasmine), spice (such as cinnamon and cardamom), and fruit scents (such as peach and plum).

Base Notes: These are the last to develop and the longest-lasting notes. Base notes give depth and solidity to the fragrance, lingering for an extended time after the top and middle notes have faded. They are usually rich, heavy, and deep scents that anchor the fragrance, preventing the lighter notes from evaporating too quickly. Common base notes include woodsy scents (like sandalwood and cedarwood), resins (such as amber and myrrh), musk, and vanilla.

In the above topology of notes, base notes may be described as “lower” than middle notes, which are lower than top notes. Conversely, top notes may be described as “higher” than middle notes, which are higher than base notes.

In some embodiments, a break period may be presented by the scent presentation system between the presentation of different scents. During this break period, no scent is dispersed. The break period can be longer following base notes than middle or top notes, and the break period after a middle note can be longer than that following a top note. The break period can also be based on the room size, number of windows, available air ducts, type of scents, or a combination thereof.

### Control Module

The scent presentation device/system may include a control module to disperse a first scent for a predetermined first duration, employ a break period after the predetermined first duration, and disperse a second scent for a predetermined second duration after the break period. In an embodiment, multiple different scents may be presented in sequence, but separated by break periods. The break period can be based on the type of scent just dispersed, how strong the scent is, and/or where it is located on a scent pyramid model.

The control module may comprise programmable logic circuit (PLC) that allows for the customization of operational sequences. The PLC may be programmed to initiate a control signal to activate a dispersion mechanism for a specified first duration.

Upon receiving a control signal, the dispersion mechanism may operate for a predetermined first duration, performing its designated function. The duration may be programmable and can be set according to the requirements of a specific application.

After operating for a first duration, the PLC may initiate a break period. This period may be of a predefined or programmable length during which dispersion mechanisms are inactive. This break period facilitates the dissipation of the first scent, ensuring it can no longer be detected (via smell).

After the break period, the PLC may generate a second control signal to activate the same or different dispersion mechanism to disperse a second scent. The same or different dispersion mechanism then operates for a predetermined second duration.

Incorporated within the PLC may be timing and sequencing logic algorithms that ensure the precise control of the activation periods and the break period. This logic is programmable to accommodate various sequences, durations, and operational requirements.

The control module may include an interface for programming and adjusting the sequence parameters, durations, and break periods. This interface may be a physical set of controls on the module or a software application communicating with the module via wired or wireless connections (e.g., over-the-air updates).

### Physical Design

The scent presentation device/system can have a plurality of reservoirs and dispersion mechanisms, each supplying a different scent. The plurality of reservoirs can also include one or more scent neutralizers that may be dispersed in the same manner as the essential oils or other scent-bearing liquids described above.

In an embodiment, liquid (e.g., oil, water-oil mixture) having a first scent is transferred into a dispersion reservoir where the dispersion mechanism is configured to disperse any liquid transferred into the dispersion reservoir. In this embodiment, the dispersion device/system can have multiple scent reservoirs and a single dispersion reservoir. In another embodiment, the scent presentation device/system can have multiple dispersion mechanisms. In this embodiment, each scent reservoir can have its own dispersion mechanism and as such a centralized dispersion reservoir is not needed. However, the dispersion system can include a combination of scent reservoirs having their own dispersion mechanisms and scent reservoirs with a shared dispersion chamber.

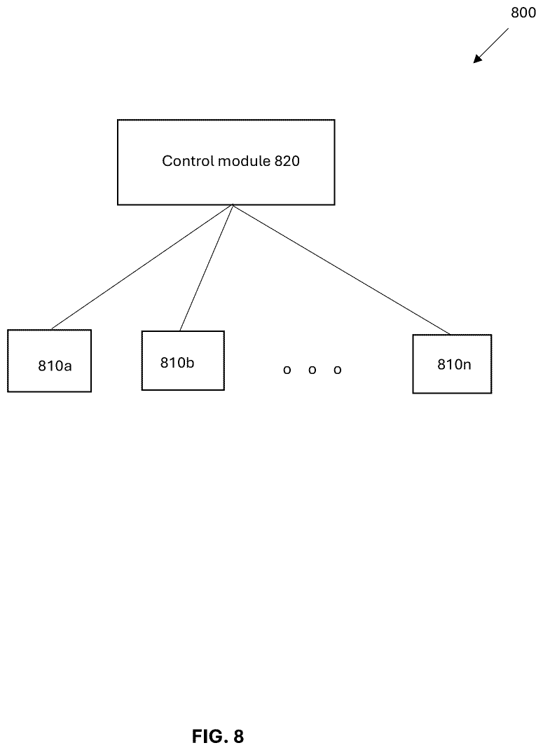

In an embodiment, shown in FIG. 8, the plurality of dispersion mechanisms 810 may be under the control of a control module 820. Each dispersion mechanism may disperse different scents. The control module may control which dispersion mechanism is operating at a given time, when it starts, when it stops, and how long a break period lasts before another dispersion mechanism, with a different scent, begins.

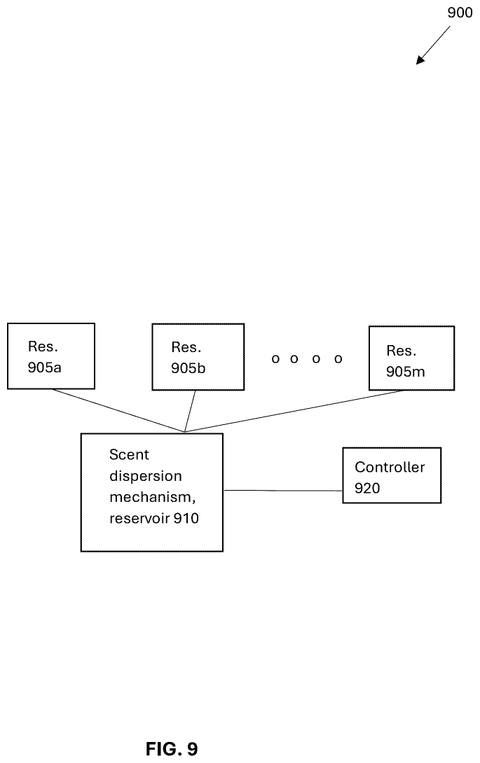

In some embodiments, the device/system can have multiple reservoirs, where the system also includes a dispersion mechanism having a dispersion reservoir configured to disperse any liquid in the dispersion reservoir. This is shown in FIG. 9. The dispersion mechanism with its reservoir (910) can be centrally located to the multiple scent reservoirs 905. In operation, liquid from one of the scent reservoirs can be transferred (e.g., pump, gravity) into the dispersion reservoir. Once the transfer process is completed, the dispersion mechanism can be activated to disperse the liquid therein. As before, a control module (here, 920) may be used to control which scent reservoir (i.e., which scent) is being used at a given time, when the dispersion of that scent starts, when it stops, and how long a break period lasts before a different scent begins.

### Further Embodiments

In some embodiments, liquid from two or more reservoirs can be transferred to the dispersion reservoir for dispersion. In this way, a unique blend of aromas can be created using two or more scented liquids.

In some embodiments, the dispersion device/system can dispense droplets of the liquid (e.g., essential oil, water-oil mixture) into a dispersion chamber where the droplets are exposed to an airstream using various means such as a fan, convection, pressure differential system, etc. The dispersion chamber may also include a dispersion plate configured to disperse or evaporate the droplets. The dispersion plate can employ aerosolization or evaporation mechanism as described herein. For example, the dispersion plate can use an ultrasonic transducer to disperse the droplets. Alternatively, the dispersion plate can use a heat source to speed up the evaporation process.

Various mechanisms can be employed to accurately disperse liquid droplets. The dispersion device/system can employ any of the droplets dispensing mechanisms described herein.

Piezoelectric droplet ejection leverages the properties of piezoelectric materials to precisely eject liquid droplets, a technique widely adopted in inkjet printing. Similarly, microfluidic devices manipulate fluids within micro-scale channels to produce uniform droplets.

### CONCLUSION

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the claims and their equivalents.

What is claimed is:

1. A scent presentation device, the device comprising:
a plurality of scent reservoirs each configured to contain a liquid;
at least one liquid dispersion mechanism configured to disperse liquid into an environment;
a control module configured to cause the at least one liquid dispersion mechanism to disperse liquid from a first scent reservoir of the plurality of scent reservoirs for a first duration, and to disperse liquid from a second scent reservoir of the plurality of scent reservoirs for a second duration.

2. The scent presentation device of claim 1 comprises a plurality of liquid dispersion mechanisms.

3. The scent presentation device of claim 2, wherein each of the plurality of liquid dispersion mechanisms is configured to disperse liquid from a different respective liquid reservoir.

4. The scent presentation device of claim 2, wherein the at least one dispersion mechanism comprises an aerosolization device.

5. The scent presentation device of claim 4, wherein the aerosolization device comprises an ultrasonic diffuser.

6. The scent presentation device of claim 4, wherein the aerosolization device comprises a nebulizer.

7. The scent presentation device of claim 6, wherein the nebulizer comprises one or more of an ultrasonic nebulizer, a jet nebulizer, or a mesh nebulizer.

8. The scent presentation device of claim 4, wherein the aerosolization device comprises an atomizer.

9. The scent presentation device of claim 2, wherein the at least one dispersion mechanism comprises a vaporizer configured to convert at least some of the liquid into gas.

10. The scent presentation device of claim 1, further comprising a dispersion reservoir having fluid communication with the plurality of scent reservoirs and to the at least one liquid dispersion mechanism.

11. The scent presentation device of claim 10, wherein the control module is further configured to cause liquid from the first scent reservoir to transfer to the dispersion reservoir, and wherein the at least one liquid dispersion mechanism is configured to disperse liquid from the dispersion reservoir for the first duration.

12. The scent presentation device of claim 11, wherein the control module is further configured to cause a second liquid from a second scent reservoir to transfer to the dispersion reservoir after the first duration is completed, and wherein the at least one liquid dispersion mechanism is configured to disperse the second liquid from the dispersion reservoir for the second duration.

13. The scent presentation device of claim 12, wherein the control module is configured to create a break period between the first and second durations, wherein the break period represents a period where there is no dispersion activity.

14. The scent presentation device of claim 13, wherein the first and second durations are of different lengths and wherein a scent presented in the first duration and a scent presented in the second duration are different.

15. The scent presentation device of claim 14, wherein the scent presented in the first duration and the scent presented in the second duration are of different categories of scents.

16. The scent presentation device of claim 15, wherein the length of the break period depends on whether the scent presented in the first duration is a base, middle, or top note.

17. The scent presentation device of claim 16, wherein the length of the break period longer following a base note scent than middle or top note scents, and the break period after a middle note scent is longer than that following a top note scent.
